(19) United States
(12) Patent Application Publication
(10) Pub. No.: US 2016/0129151 A1
(43) Pub. Date: May 12, 2016

(54) PROTEIN BASED MATERIALS

(71) Applicant: Case Western Reserve University, Cleveland, OH (US)

(72) Inventors: Matthew J. Fullana, Windsor, CT (US); Daniel J. Brannum, Xenia, OH (US); Gary E. Wnek, Cleveland, OH (US)

(21) Appl. No.: 14/534,869

(22) Filed: Nov. 6, 2014

(51) Int. Cl. A61L 27/24; A61L 24/00; A61L 27/56; A61L 24/10

(52) U.S. Cl. CPC A61L 27/24; A61L 24/102; A61L 24/0036; A61L 27/56; A61L 2400/06

(57) ABSTRACT

Gels and films can be formed from protein dissolved into a benign solvent that comprises alcohol, water, and salt. In one example, the protein can be collagen. In one example, the benign solvent can include a water to alcohol ratio of between ninety-nine-to-one and one-to-ninety-nine by volume, a salt concentration between zero moles per liter and the maximum salt concentration soluble in water, and a protein amount of between near zero percent and about 25 percent by weight as compared to the mixture of water and alcohol. Once the protein is dissolved in the benign solvent, secondary processing steps can be conducted to form protein based bioadhesives, gels, and films with desirable physical properties. Additional process steps can include washings that improve the properties of the protein structures.

### CROSS-REFERENCE TO RELATED APPLICATIONS

This application is a continuation-in-part of U.S. patent application Ser. No. 13/430,562, titled “Controlled Cross-Linking Processing of Proteins,” filed on Mar. 26, 2012, which is a continuation-in-part of U.S. patent application Ser. No. 12/571,043, now U.S. Pat. No. 8,318,903, titled “Benign Solvents for Forming Protein Structures,” filed on Sep. 30, 2009, which claims priority to U.S. Provisional Patent Application Ser. No. 61/194,685, titled “Electrospinning of Fiber Scaffolds,” filed on Sep. 30, 2008. This application claims priority to and the full benefit of the foregoing referenced patent applications, which are incorporated by reference as if fully rewritten herein. This application further claims priority to and the full benefit of U.S. Provisional Patent Application Ser. No. 61/900,506, titled “Protein Based Materials,” which was filed on Nov. 6, 2013, which is incorporated by reference as if fully rewritten herein.

### TECHNICAL FIELD

The disclosed description relates generally to a process for forming protein based structures and materials, and more specifically, relates to processes for forming protein based bioadhesives, gels, and films that exhibits desirable physical properties.

### BACKGROUND

Materials, products and devices constructed from man-made materials can be implanted into, applied onto, or otherwise used with a human body to benefit the human by, for example, treating injuries, diseases, and other conditions of the human body. The materials chosen for such products or devices can be important with regard to how successfully a product or device treats conditions of the human body. For instance, the compatibility of a material with the human body can determine if the product or device can be positioned on or in the human body. Products or devices can be made from synthetic materials. However, if the synthetic material is dissimilar to human tissue, the success of the product or device can be limited. Products and devices constructed from naturally occurring materials such as proteins can provide biocompatible products or devices for application onto or implantation or injection into the human body to treat conditions of the human body.

### SUMMARY

Gels and films can be formed from protein dissolved into a benign solvent that comprises alcohol, water, and salt. In one example, the protein can be collagen. In one example, the benign solvent can include a water to alcohol ratio of between ninety-nine-to-one and one-to-ninety-nine by volume, a salt concentration between zero moles per liter and the maximum salt concentration soluble in water, and a protein amount of between near zero percent and about 27 percent by weight as compared to the mixture of water and alcohol. Once the protein is dissolved in the benign solvent, secondary processing steps can be conducted to form protein based bioadhesives, gels, and films with desirable physical properties. Additional process steps can include washings that improve the properties of the protein structures.

### BRIEF DESCRIPTION OF THE DRAWINGS

Objects and advantages together with the operation of the invention may be better understood by reference to the detailed description taken in connection with the following illustrations, wherein:

FIG. 1 is a photograph showing a vial of protein dissolved in a benign solvent.

FIG. 2 is a photograph showing the formation of a protein precipitate upon the addition of alcohol to the vial of FIG. 1.

FIG. 3A-D are photographs depicting the process of forming a protein-based gel from a protein-based bioadhesive.

FIG. 4A-B are photographs depicting two glass slides adhered by a protein-based bioadhesive.

FIG. 5 is a photograph of protein fibers on a glass slide.

FIG. 6 are photographs of a protein-based gel submerged in water for various durations of time.

FIG. 7 is an SEM image of a lyophilized foam generated from a protein-based gel.

FIG. 8 is another SEM image of a lyophilized foam generated from a protein-based gel.

FIG. 9 is a flowchart illustrating the process of forming a protein-based gel.

FIG. 10A-C are photographs illustrating steps in the process of forming a protein film.

FIG. 11 is a graph illustrating the results of circular dichroism.

FIG. 12 is a graph illustrating the results of Fourier Transform Infrared Spectroscopy.

FIG. 13A-B are photographs of the effect on protein structured submerged in water.

### DETAILED DESCRIPTION

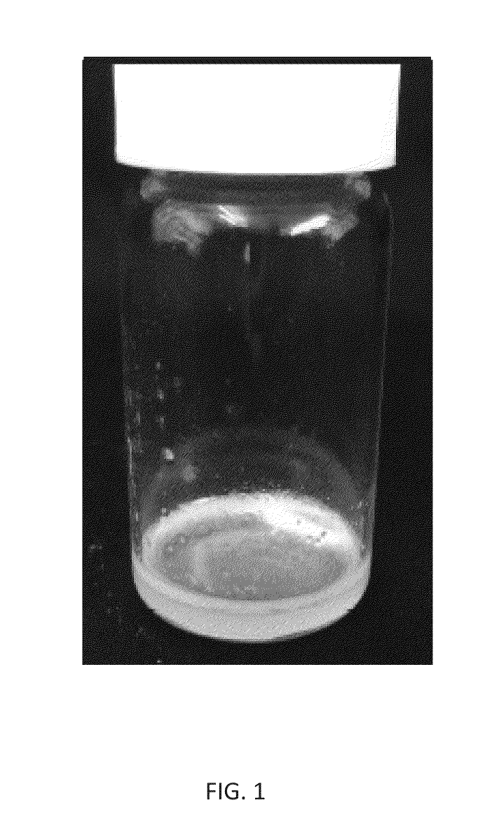

The apparatus and methods disclosed in this document are described in detail by way of examples and with reference to the figures. It will be appreciated that modifications to disclosed and described examples, arrangements, configurations, components, elements, apparatuses, methods, materials, etc. can be made and may be desired for a specific application. In this disclosure, any identification of specific shapes, materials, techniques, arrangements, etc. are either related to a specific example presented or are merely a general description of such a shape, material, technique, arrangement, etc. Identifications of specific details or examples are not intended to be and should not be construed as mandatory or limiting unless specifically designated as such. Selected examples of apparatus and methods for forming protein materials and structures from a solution of protein dissolved in a benign solvent are hereinafter disclosed and described in detail with reference made to FIGS. 1-13.

Naturally occurring materials are good candidates for products and devices that are intended for use with biological material such as human and animal tissue. One category of materials that can be compatible with the biological material is natural polymers such as proteins. Examples of such biocompatible proteins include, but are not limited to, collagens, gelatin, elastin, fibrinogen, silk, and other suitable proteins. Such proteins can be used to form protein structures for implantation into, injection into, or application onto a human body. Other materials that are generally biocompatible are polysaccharides such as hyaluronic acid, chitosan, and derivatives of starch and cellulose such as hydroxypropyl methyl cellulose phthalate, deoxyribonucleic acid (DNA), and ribonucleic acid (RNA).

One example of a protein structure that can be useful for applications either in or on the human body are bioadhesives and gels made from collagen. As will be described herein and in U.S. Pat. No. 8,318,903 and U.S. patent application Ser. No. 13/430,562, both of which are incorporated herein, collagen can be dissolved in a benign solvent and further processed to form usable structures such as gels, foams, films, fibers (including electrospun fibers), and the like.

One method of forming a protein structure begins with dissolving a protein such as collagen in a solvent. Once dissolved, the protein can be extracted from the solvent and organized into a protein structure. One common solvent is 1,1,1,3,3,3 hexafluoro-2-propanol (HFP). However, any protein structure produced using such a solvent can have limited usefulness because of health concerns. For example, the United States Food and Drug Administration (FDA) has strict guidelines as to the amount of HFP allowed in a device, product, or material intended for use with the human body. Because of strict FDA guidelines and general health concerns, using a solvent with benign characteristics for dissolving proteins or other biocompatible materials can yield biocompatible structures for use with a human body. Generally, a benign solvent is a solvent that either reduces health risks to a human body or is of minimal or no risk to the health of a human body. Such benign solvents are described and disclosed herein.

One example of a benign solvent for dissolving protein comprises water, alcohol, and salt. The protein can be a Type I collagen, the alcohol can be ethanol, and the salt can be sodium chloride (NaCl). The association between water molecules, salt, and alcohol creates a complex structure in which proteins such as collagen are substantially soluble. Collagen is insoluble in most solvents because of interpeptide interaction. Collagen is substantially soluble in suitable water-alcohol-salt benign solvents because the properties of the solvents screen interpeptide interaction that usually results in insolubility of collagen. For example, the electrostatic interaction between the salt and the carbonyl group of the hydrophilic part of collagen and the hydrophobic interaction between the hydrocarbon chain of ethanol and the hydrophobic part of collagen can screen such interpeptide interaction. In general, any molecule or complex that exhibits a hydrophilic part and a hydrophobic part spaced by approximately the same distance as the hydrophilic part and hydrophobic part of the collagen molecule can dissolve collagen. Although examples described herein include Type I collagen, it will be understood that all collagens—Type II, Type III, and so on—can be dissolved in a benign solution and result in a protein structure for use with human tissue. In one example, the protein dissolved in a benign solution can be a semed S bovine dermis-derived collagen powder comprising 95 percent type I and 5 percent Type III.

Generally, in suitable water-alcohol-salt solvents, the ratio of water to alcohol can range from a volume ratio of about 99:1 to about 1:99, the salt concentration can range from 0 moles per liter (M) to the maximum salt concentration soluble in water, and the amount of protein by weight (as compared to the solvent) can range from near 0 percent to about 25 percent. In one example, the benign solvent comprises about a one-to-one ratio of water to ethanol and a salt concentration of about 3 M NaCl. Collagen is dissolved in such a solvent until the solution reaches about 17.5 percent collagen by weight. In another example, the solution comprises semed S (principally collagen type I with a ca. 5 percent collagen type III) dissolved in a solvent comprising phosphate buffered saline (PBS) buffer and ethanol, where the buffer to ethanol ratio of about one-to-one by volume. The saline concentration in the PBS buffer can range from 5× to 20×. The collagen concentration can be, for example, about 17.5 percent as compared to the total weight of the PBS/ethanol solvent. In yet another example, the protein dissolved in the solvent can be gelatin. The solvent can comprise a PBS buffer with a salt concentration of 10× mixed with ethanol at a one-to-one ratio by volume. Gelatin can be dissolved until the amount of gelatin by weight is about 16 percent by weight.

When protein has been dissolved in a suitable water-alcohol-salt solvent to form a protein solution, suitable processing methods can be used to extract protein from the solution and form protein structures. As previously discussed, such protein structures can be implanted into, injected into or applied onto the human body to affect treatment of a condition.

As will be understood, the pH level, temperature, type of collagen, and type and concentration of salt all influence the structure of collagen in the protein solution. For example, at low collagen concentrations and a pH level of about 7.4, the transition temperature of crystalline polymer to random coil polymer is about 45 degrees Celsius. The transition temperature can be independent of salt concentration for potassium chloride (KCl) and NaCl. There is a progressive decrease in precipitation of collagen, that is to say that collagen becomes more soluble, as more salt is added. Addition of salt results in destabilization of the precipitated collagen while the ionic strength increases with salt additions. Collagen solubility can increase even if it appears that the crystalline structure of collagen is maintained upon addition of salt.

Alcohol affects the solubility of collagen in the buffer and ethanol solution. Alcohol and collagen interaction is moderated by hydrocarbon chain length, with alcohol disrupting internal hydrophobic interactions in the collagen. With increased alcohol concentration, there is a progressive increase in molar destabilization of the crystalline collagen precipitated in an alcohol and potassium acetate buffer mixture at an acidic pH, for example, a pH of about 4.8. For single collagen molecules, structural stability is primarily a function of interpeptide hydrogen bonding and chain rigidity.

The addition of salt promotes the solubility of collagens. Hydrogen bonding between the hydrophilic part of collagen and water molecules can be too weak to break the interpeptide interaction, and the stronger electrostatic forces induced by salt in aqueous media may be necessary. The combination of both electrostatic and hydrophobic forces appears to interact strongly enough with the collagen chain to substantially dissolve the collagen in a mixture of ethanol and PBS buffer with an about one-to-one ratio when a salt concentration is at least about 5× in the buffer.

The concentration of salt and ethanol can affect the solubility of collagens in water. Collagen can be generally insoluble at about 17.5 percent by weight in either PBS (20×) or ethanol. However, when a small amount of ethanol is added into PBS (20×) buffer to form a PBS (20×) to ethanol volume ratio of about nine-to-one, the collagen substantially dissolves into this mixture. By adding more ethanol into PBS (20×) buffer (that is, the volume ratio decreases from about nine-to-one to about seven-to-three to about one-to-one) there is generally no affect on the solubility of collagen. The collagen remains substantially soluble. However, when the PBS (20×) to ethanol volume ratio is reduced to three-to-seven, collagen is generally no longer soluble. Furthermore, the salt concentration affects the solubility of collagen when the water to ethanol volume ratio is held constant at about one-to-one. The salt concentration in 5×, 10× and 20× PBS buffer is sufficient to substantially dissolve collagen in the mixture solution.

In one example, as illustrated by FIG. 1, semed S collagen comprising 95 percent Type I collagen and 5 percent Type III collagen can be dissolved in a benign solvent. In one example, the collagen can be a semed S bovine dermis-derived collagen powder. The benign solvent can comprise a one-to-one ratio by volume of phosphate buffered saline 20× (PBS 20×) and absolute ethanol, which forms a slightly hazy solvent. Approximately 17.5 percent by weight of the semed S collagen powder is added to the solvent and stirred until the collagen is dissolved, the result is as shown in FIG. 1. In one example, the collagen is dissolved after approximately 10-15 minutes of stirring. As shown, the result is a slightly viscous solution that is translucent-to-clear in appearance.

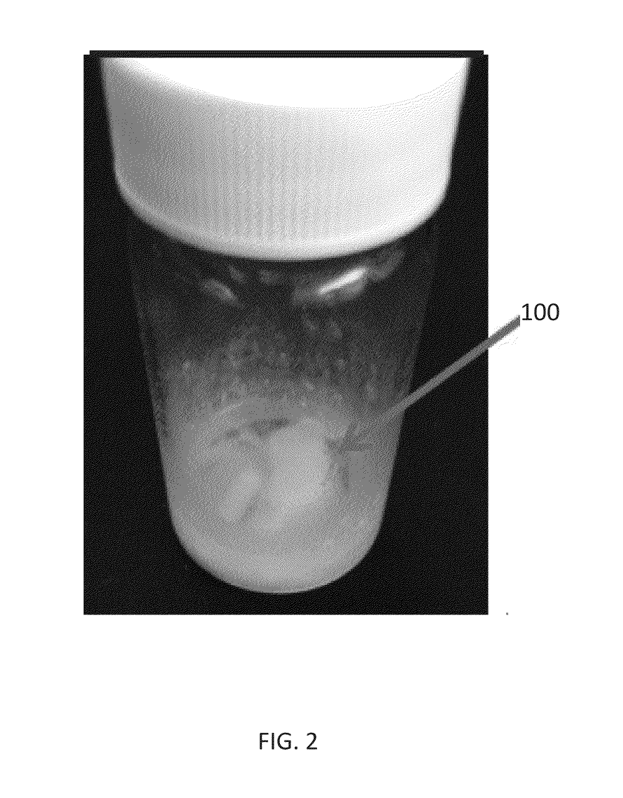

The solution as shown in FIG. 1 can be transformed into a biphasic system with the addition of absolute ethanol to the solution. In one example, a biphasic system is formed by titrating a two-to-one ratio (by volume) of absolute ethanol to starting solution into the dissolved collagen system. This step can be accompanied by the stirring of the collagen system. At a critical ethanol concentration, the collagen begins to precipitate out of solution. Continued titration results in the agglomeration of precipitated collagen and the formation of clusters. Such clusters 100 are illustrated in FIG. 2. As shown, the clusters 100 form as a white globular material, leaving a layer of ethanol in the vial. The ethanol layer can be decanted leaving behind the clusters 100. Upon inspection, the clusters 100 form a sticky, white collagen-based substance with adhesive properties. This collagen-based substance can be used as an adhesive that is compatible with the human body. Hereinafter, this collagen-based substance will be referred to as a bioadhesive.

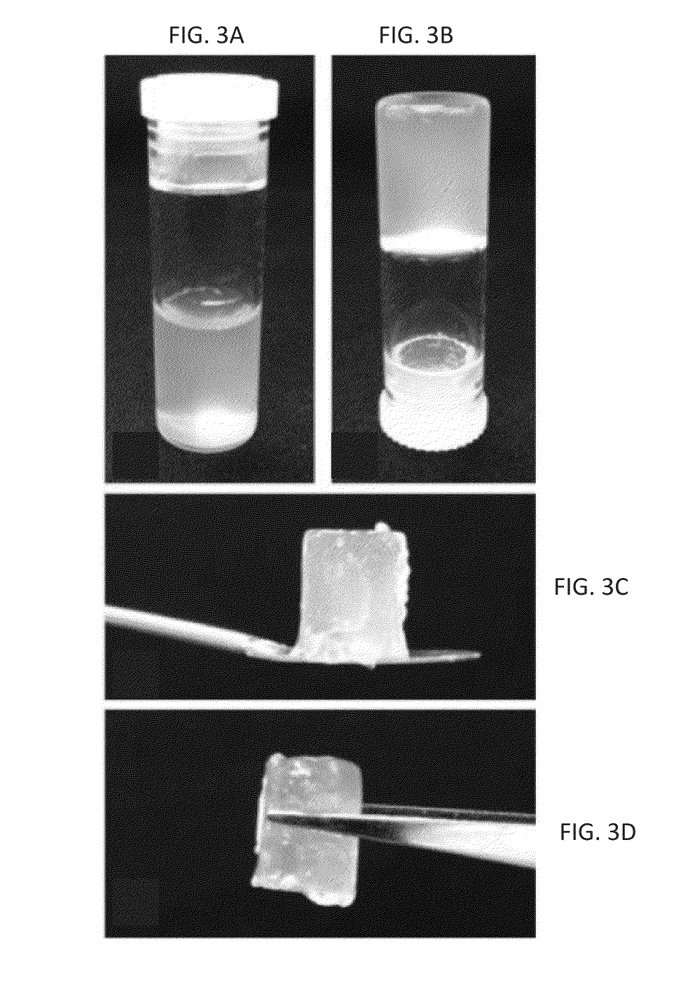

The collagen-based bioadhesive as described above can undergo additional processing so as to physically crosslink the collagen-based bioadhesive to form a collagen-based gel. In one example, the collagen-based bioadhesive is prepared as described above, and 270 mg of the collagen-based bioadhesive is added to 1 mL of HPLC-grade water. The mixture is stirred for approximately five minutes until all the collagen-based bioadhesive is dissolved and forms a homogeneous solution. When the collagen-based bioadhesive dissolves in the solvent, the solution appears as slightly hazy. After the solution is allowed to remain undisturbed for two hours, the collagen forms a soft-set gel. FIGS. 3A-3D illustrate the described process. FIG. 3A illustrates the collagen-based gel in a glass vial. The solution is allowed to remain undisturbed for two hours. FIG. 3B illustrates the vial inverted, with the collagen-based gel remaining at the “bottom” of the vial and separate from the remaining solution (i.e., when inverted, the “bottom” of the vial is shown at the top of FIG. 3B). When the collagen-based gel is removed from the vial, it is apparent that physical crosslinking has occurred in the gel. The gel has structural integrity and is not fluid. FIG. 3C illustrates the gel resting on the edge of a spatula. FIG. 3D illustrates that the gel can be gripped and held using forceps without damaging the gel.

In another example, collagen-based bioadhesive can be physically crosslinked using hyaluronic acid. A collagen-based bioadhesive is prepared again as described above and 270 mg of collagen-based bioadhesive was added to a 1 mL mixture of hyaluronic acid and water, where the ratio is 5 mg hyaluronic acid per mL water. The mixture is stirred for about five minutes until a homogeneous solution is achieved. Once all adhesive was dissolved, a slightly hazy solution is produced. When the solution remains undisturbed for approximately 15 minutes, a soft-set and physically crosslinked collagen-based gel is produced. The collagen-based gel as described above can undergo additional processing to form a collagen-based foam. In one example, a lyophilization process is used to form a collagen-based foam. The collagen-based gel as described above is immersed in liquid nitrogen for approximately five minutes until it is frozen. The frozen gel is placed into a lyophilizer with a condenser temperature set to −80 degrees Celsius. The initial shelf temperature was set to −70 degrees Celsius and heated to 25 degrees Celsius at a rate of 0.5 degrees Celsius per minute under five microbar vacuum. Such a process resulted in a collagen-based porous foam.

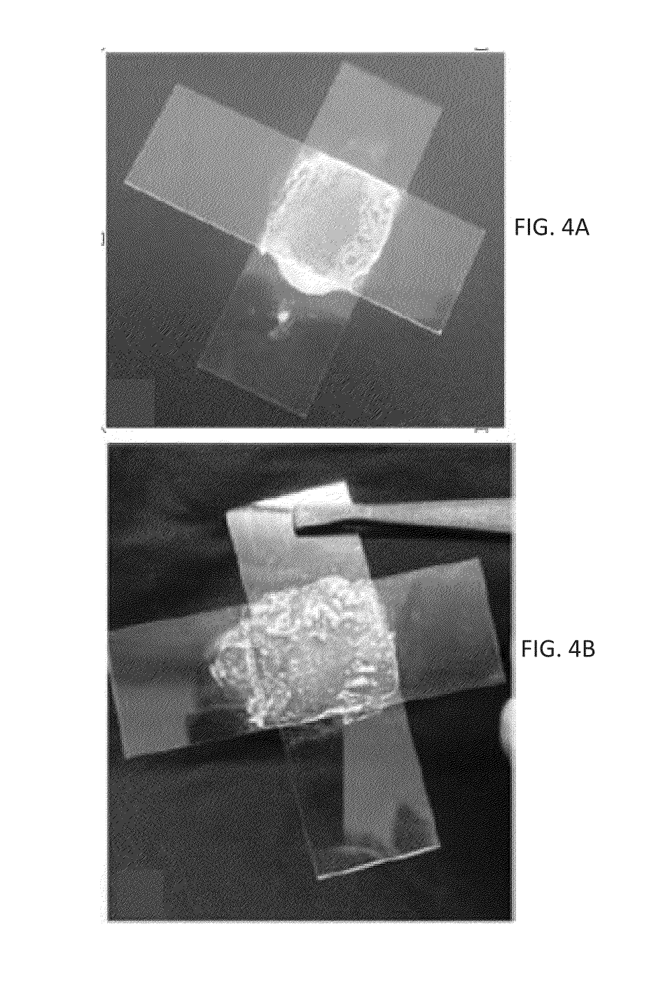

The collagen-based gel as described herein has a number of valuable applications. For example, the collagen-based bioadhesive gel can have useful application in the medical field. The preparation of this adhesive is novel, and it does not require the use of any harsh organic solvents. The components are collagen, water, phosphate buffer, and ethanol. To test the adhesive properties of the collagen-based bioadhesive, the bioadhesive was spread over a glass microscope slide. A second slide was placed on top to form a sandwich, in a T-configuration, as illustrated in FIG. 4A. The slides were left at room temperature for two hours. After the two hour period, a manual test showed that the slides were firmly adhered to each other. After 24 hours, the slides were immersed in a water bath. After an hour, the collagen-based bioadhesive showed no signs of dissolution. The slides remained well adhered, see FIG. 4B. After 2 days, a manual test indicate that the slides showed no relative rotation beyond approximately one to two degrees, and the slides were still firmly adhered to one another. The slides could be manually separated after subjecting the slides to manual impact by dropping the slides from approximately 2 feet onto a rigid surface. Once separated, the slides could be readhered to one another using the existing bioadhesive residue on the slides.

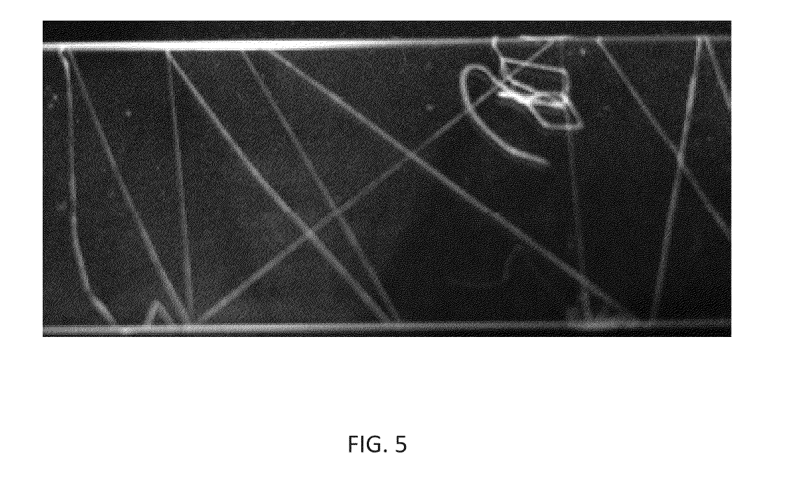

In addition to forming an adhesive, the collagen-based bioadhesive can be drawn into fibers. FIG. 5 illustrates the collagen-based bioadhesive manually drawn into fibers and attached to a glass microscope slide. Furthermore, if the collagen-based bioadhesive is allowed to dry, it will form a thin film or coating that is resistant to dissolution in water.

The collagen-based bioadhesive described herein is resistant to dissolution in water beginning approximately two hours after it has precipitated from a solution of collagen and benign solvent. However, for approximately two hours after the bioadhesive has initial precipitation from the benign solution, the bioadhesive is water soluble. During this approximately two hour period, the bioadhesive can be mixed with water to form a solution that can be injected into a human body by, for example, a syringe. The ratio of bioadhesive to water can be selected to achieve any desired viscosity.

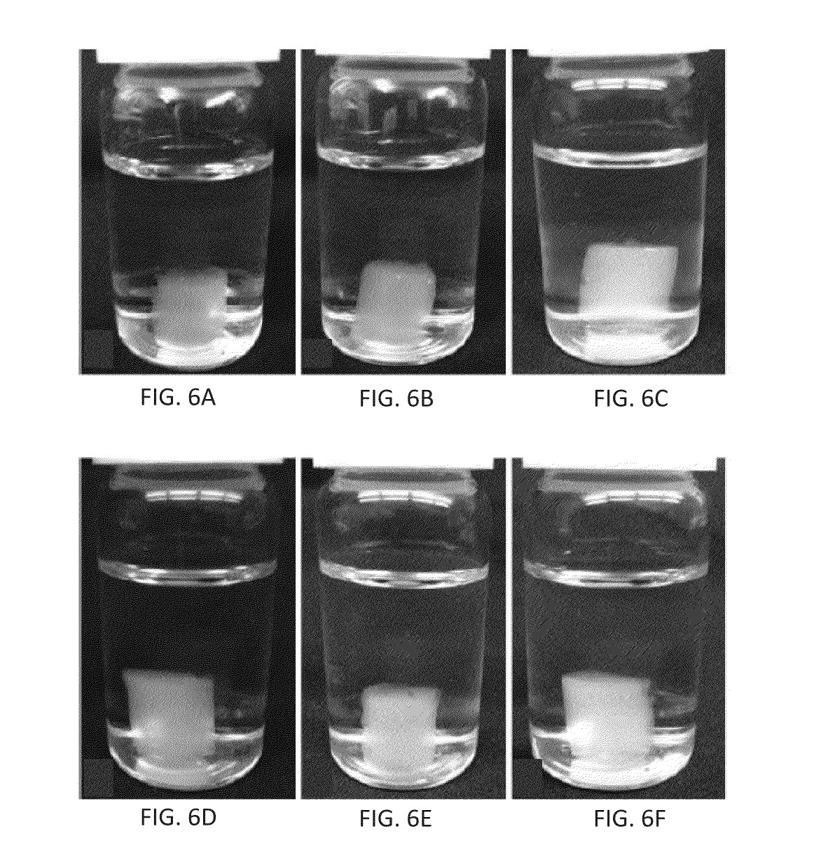

As demonstrated in the photographs shown in FIG. 6, the collagen-based gel as described herein does not dissolve in water. FIG. 6A shows the gel submerged in water at zero hours (i.e., initial immersion in water). FIG. 6B shows the gel submerged in water after one hour. FIG. 6C shows the gel submerged in water after three hours. FIG. 6D shows the gel submerged in water after four hours. FIG. 6E shows the gel submerged in water after 24 hours. FIG. 6F shows the gel submerged in water after 96 hours. The ability of the gel described here to withstand submersion in water (over at least four days) without the addition of any crosslinking agent is indicative that physical, rather than chemical, crosslinking has occurred.

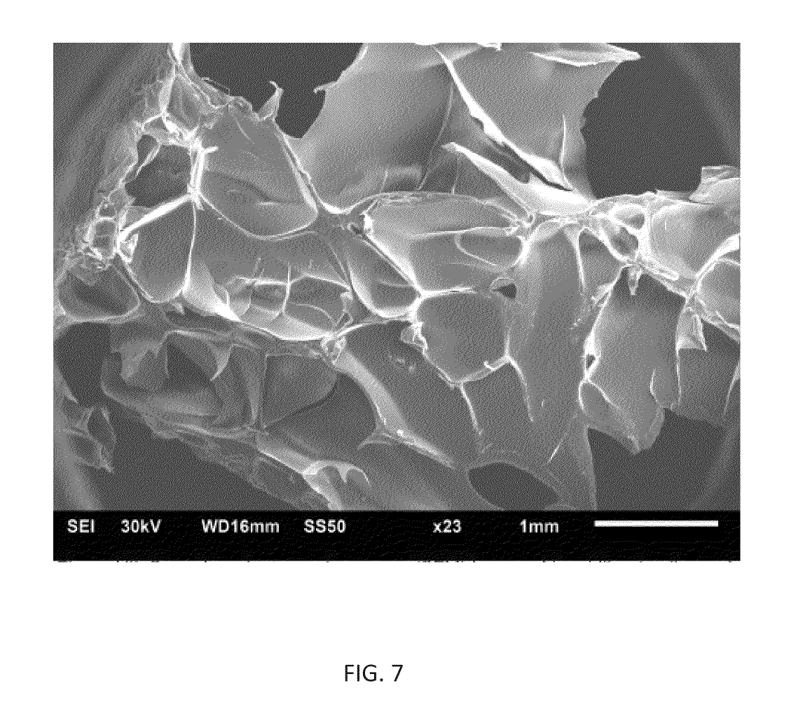

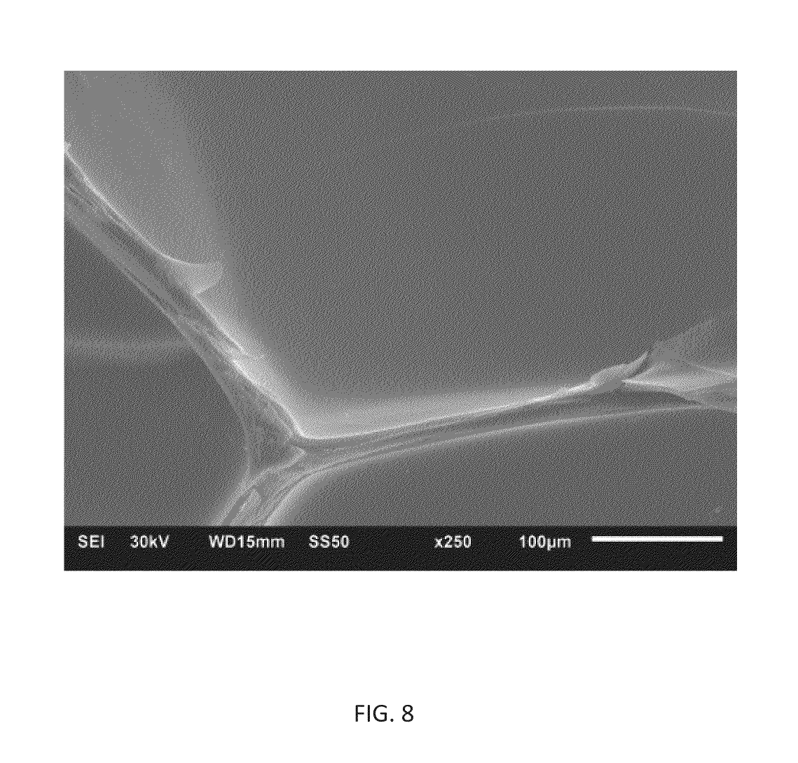

Scanning electron microscopy (SEM) images of the lyophilized foam generated from a collagen-based gel is shown in FIGS. 7 and 8. As can be seen, the lyophilized foam samples include relatively large pores that are approximately 1 mm in diameter in one example.

In another example, the collagen solutions as described herein can also be injected in a human body to achieve desired results. For example, the collagen gel precursor (the solution prior to gelation after 2 hours) is capable of being injectable into a human body. Such injections can be used in fields such as plastic surgery as, for example, a potential dermal filler. The formulations as described herein have the advantage of becoming insoluble after a short time period. Such insolubility can prevent rapid reabsorption of the collagen-based substance. It will be understood that reabsorption can be a problem with dermal fillers.

The collagen-based gels produced using hyaluronic acid demonstrate the ability to incorporate water-soluble materials (such as hyaluronic acid) into these novel collagen-based gels and can be useful for a variety of biomedical applications. One example is reconstruction of the extracellular matrix of skin. This matrix is composed primarily of collagen and supporting polysaccharides and fibrous proteins. One common polysaccharide found in the extracellular matrix of load-bearing joints is hyaluronic acid, where it serves as a cushion to surrounding tissues.

In other applications, the collagen-based gel or a solution of collagen-based bioadhesive and water can be used in a variety of biomedical applications. In one example, the collagen-based gel can be used as a temporary solution to stop bleeding at a puncture wound in a human body, such as a bullet wound. In such an example, the gel can be formed to an appropriate desired size and inserted into the wound. The general presence of the gel along with the hemostatic properties of collagen can stop the bleeding of a wound until more substantial medical assistance can be rendered. In another example, films made from the bioadhesive can be used to cover open wounds on a body. The films can prevent dirt and infections from entering the wound along with promoting healing. In another example, a drug or other healing agent can be inserted in a collagen-based gel, where the gel serves as a delivery mechanism for the drug or healing agent. In one example, a peptide that promotes bone growth can be inserted into a gel. The gel can be positioned on or near a bone, and the peptide can be delivered to the bone based on the proximity of the gel.

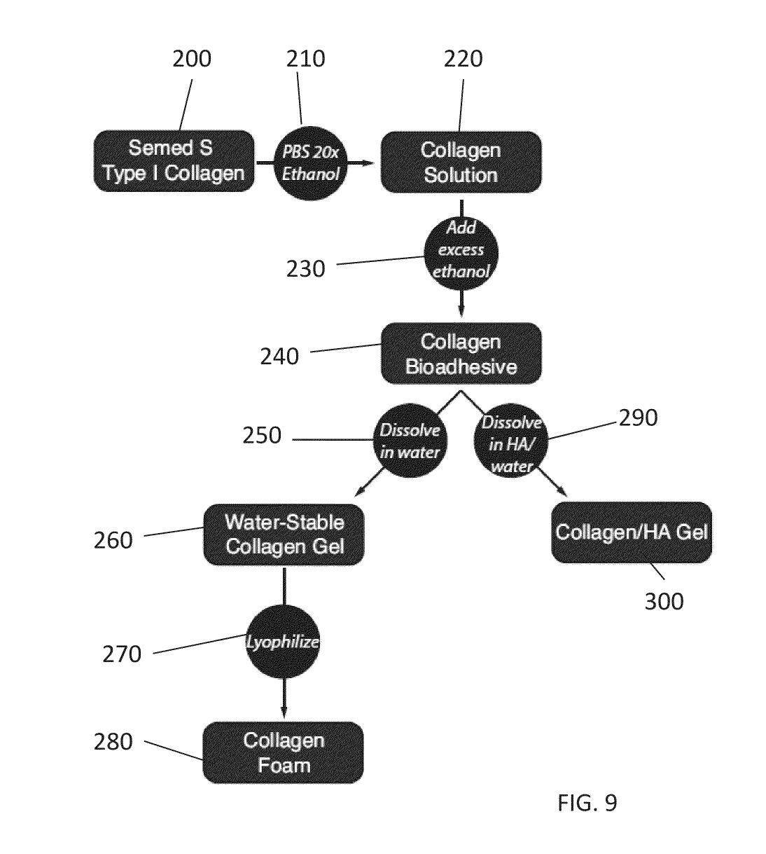

For clarity, FIG. 9 illustrates a flowchart of processes for forming gels as described herein. At step 200, collagen is provided. As described herein, the collagen can be a semed S Type I collagen, or a combination of semed S Type I and Type III collagen powder. At step 210, the collagen is added to a solvent of water, alcohol, and salt, which forms a collagen solution at step 220. The solvent can be phosphate buffered saline 20× (PBS 20×) and absolute ethanol combined at a volume ratio of 1:1. The collagen can be added at to the solvent at a ratio of about 17.5 percent weight. The solution can be stirred until the collagen is dissolved.

A biphasic system was created by adding excess ethanol (step 230). Excess ethanol can be added by titrating a 2:1 ratio (by volume) of absolute ethanol to the collagen solution. In the presence of the excess ethanol, the collagen precipitates out of the solution and forms a sticky, white collagen bioadhesive (step 240). The ethanol phase can be decanted, leaving behind the collagen-based bioadhesive.

As illustrated in FIG. 9, the bioadhesive can undergo alternative processing. One alternative is to add the bioadhesive to water (step 250). The water can be HPLC-grade water (water volume equivalent to volume of starting solution). The mixture can be vortexed or stirred for approximately 5-10 minutes until homogeneous. Once all adhesive was dissolved, the mixture is allowed to rest undisturbed for 2 hours. As described above, this process can result in a water-stable collagen gel (step 260). The collagen gel can subsequently be lyophilized (step 270), resulting in a collagen foam (step 280). As described earlier, the process of lyophilization can include the gel being immersed in liquid nitrogen for five minutes until frozen. The frozen gel can then be placed into a lyophilizer with a condenser temperature set to −80° C. The initial shelf temperature was set to −70° C. and heated up to 25° C. at 0.5° C./min under 5 microbar vacuum, producing a porous foam.

Another alternative is to process the collagen bioadhesive to form a collagen-hyaluronic acid gel. At step 290, the bioadhesive was added to a solution of hyaluronic acid and water. The concentration can be 5 mg of hyaluronic acid per mL water. The mixture can be vortexed or stirred for approximately 5-10 minutes until homogeneous. The mixture is allowed to rest undisturbed. After approximately 15 minutes a soft collagen-hyaluronic acid gel is produced (step 300).

Collagen gels can be cross-linked during the formation process. In one example, a crosslinking solution consisting of 200 mmol/L N-(3-dimethylaminopropyl)-N′-ethylcarbodiimide hydrochloride (EDC) and 400 mmol/L N-hydroxysuccinimide (NHS) is prepared in ethanol. The crosslinking solution is used in the preparation of a benign solvent mixture of a 50:50 volume ratio of crosslinking solution/PBS 20×. Collagen is added to the benign solvent mixture. As described herein, the collagen can be bovine dermis-derived, semed S collagen, principally 95 percent type I and 5 percent type III. The collagen can be added until the mass fraction is approximately 17.5 percent. The solution can be stirred until completely dissolved. In one example, the stirring process lasts approximately 10 minutes. The solution can be left undisturbed at room temperature to form a cross-linked gel.

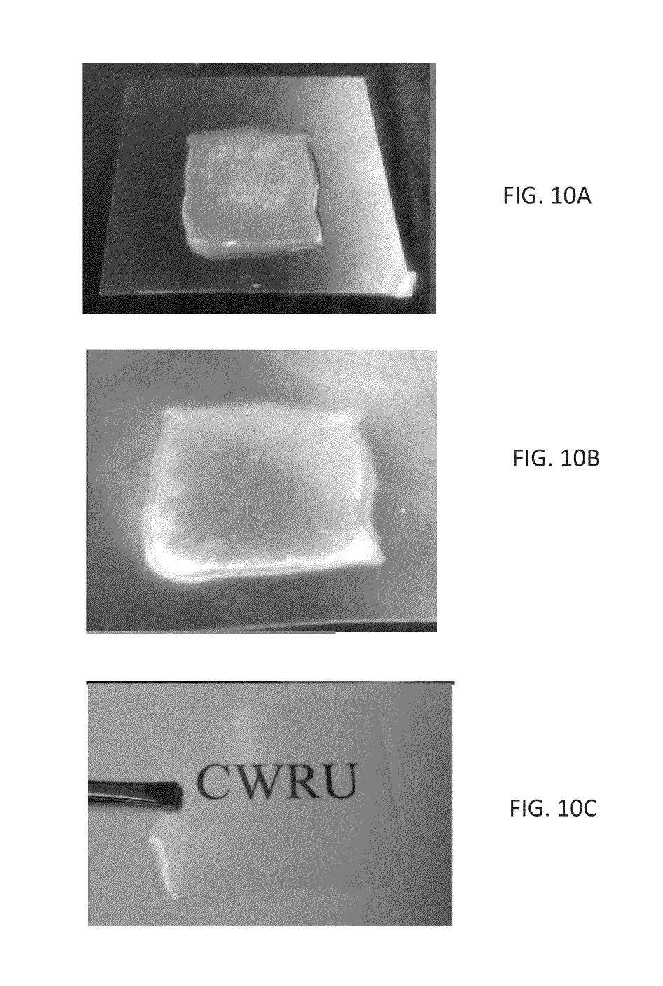

The collagen gels can also be formed into films. In one example, a collagen gel solution can be prepared as described above. Prior to gelation, the solution can be poured evenly onto a flat surface and allowed to dry under ambient conditions for approximately three hours, producing a cloudy, flexible, yet durable film. Using a 0.5 mL of collagen gel solution, a film can be produced that measures 6 cm×6 cm with a 10 μm thickness. Salt that is included in the film due to the use of PBS 20× can be removed from the film by performing washes with water. It has been shown that multiple washes (such as three washes for example) can remove most of the salt content. It has been found that multiple water washings have no impact on the mechanical characteristics of the film. FIGS. 10A-C illustrate a portion of the process for forming a film. FIG. 10A illustrates the liquid phase of the gel poured on a substrate to dry. FIG. 10B illustrate the collagen film dried on the substrate. FIG. 10C illustrates the collagen film after multiple washings with water. As illustrated in FIGS. 10A-C, the collagen film, which begins as a cloudy film, gains a transparent property after washing.

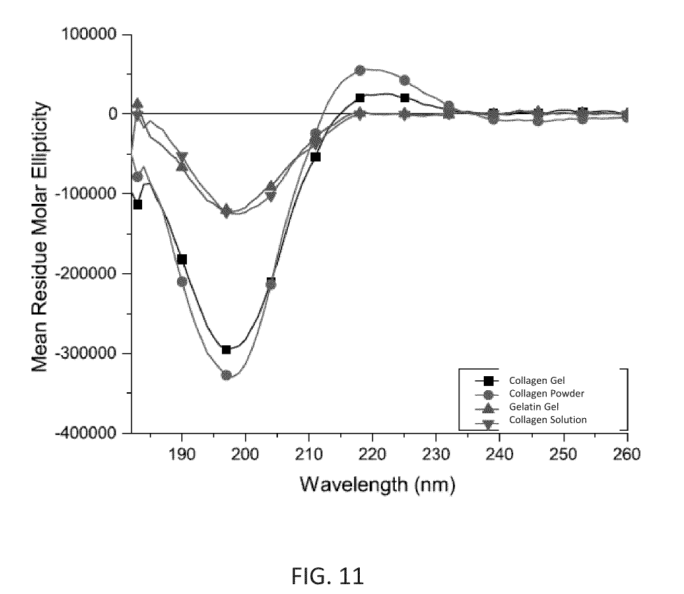

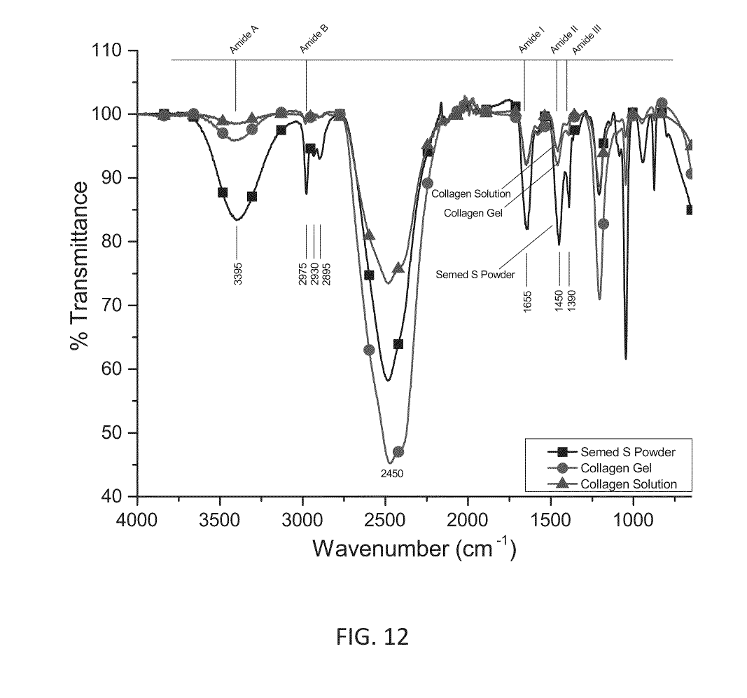

To investigate the structure of the collagen gel formed using processes described herein, circular dichroism and Fourier Transform Infrared Spectroscopy (FTIR) were performed on the collagen gel. FTIR and Circular dichroism was also performed on other protein structures so that comparisons could be made with the collagen gels. As will be appreciated, circular dichroism and FTIR can be useful in determining the physical properties and secondary structure of proteins that have been subject to recombinant processes. Four samples were examined using circular dichroism: semed S collagen powder, a collagen solution as described herein, a collagen gel as described herein, and a control gelatin gel (see FIG. 11). The semed S power, the collagen solution, and the collagen gel were also examined using FTIR (see FIG. 12).

For the circular dichroism experiments the materials were dissolved in an aqueous solution containing a 0.1 percent volume fraction of acetic acid containing a collagen concentration of 0.1 mg/mL. The solutions were filtered using a 0.22 μm syringe filter prior to circular dichroism measurement and degassed to remove any dissolved oxygen. Spectra were obtained at 25° C. over a wavelength range of 180-260 nm using a bandwidth of 0.5 nm using an AVIV model 215 circular dichroism spectrometer. A quartz cell with a 1 mm path length was used. A sample containing a volume fraction of 0.1 percent acetic acid was used as the baseline. The reported spectra were obtained by averaging three scans for each sample.

As illustrated in FIG. 11, the results of the circular dichroism shows the presence of α-helical structure in the collagen powder as evidenced by the large positive peak at 220 nm. After dissolution in the PBS/ethanol solution (i.e., the collagen solution), all α-helical structure is lost. This closely resembles the gelatin gel sample, which is known to be devoid of α-helical structure. Once the collagen gel is formed, approximately 60 percent of the α-helical structure is recovered.

For the FTIR analysis, semed S collagen powder was dissolve in deuterium oxide at a concentration of 10 mg/mL. Deuterium oxide was selected as the solvent since peaks from water would overlap with amide peaks necessary for functional group analysis. To prepare a collagen gel for FTIR analysis, PBS 20× was prepared using deuterium oxide to produce “deuterated PBS 20×.” The collagen gel was then made using the “deuterated PBS 20×.” The collagen solution was also prepared using “deuterated PBS 20×.” The semed S solution, collagen solution, and collagen gel were analyzed using an Agilent Cary 630 FTIR spectrometer. The amide II peak is frequently used to examine protein secondary structure. As illustrated in FIG. 12, the collagen solution shows the lowest intensity of the amide II peak, while the collagen gel shows an increase in intensity, consistent with the circular dichroism results.

The production of a collagen-based bioadhesive has value in applications such as medical technology. The preparation of this bioadhesive is novel in that it does not require the use of any harsh organic solvents (uses only collagen, water, phosphate buffer, and ethanol). Crosslinking methods are frequently employed to provide water stability and mechanical strength to collagen-based materials, such as nanofibrous scaffolds and gels. The collagen gels described here can be physically crosslinked.

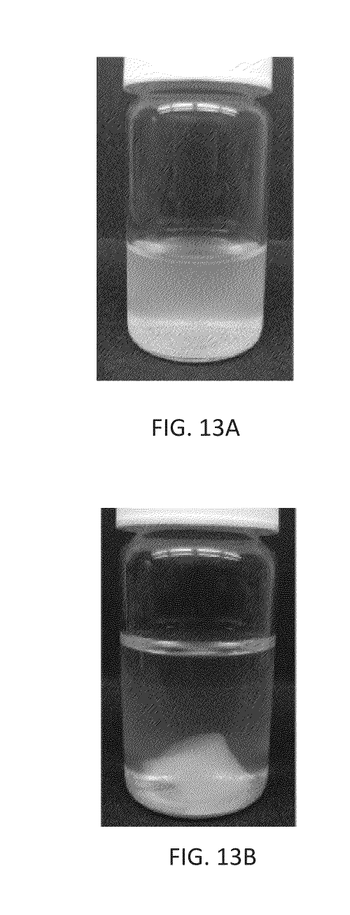

The gels formed with methods disclosed herein exhibit better water stability than a similarly prepared collagen gel crosslinked using a carbodiimide crosslinking system. As shown in FIG. 13A, following immersion in water for 138 days, a chemically crosslinked collagen gel succumbed to hydrolysis reactions, generating a cloudy solution of collagen and water. As shown in FIG. 13B, a collagen gel prepared using the method described here remained intact, showing that this system shows improved stability in water which is highly favored for biomedical applications.

Analysis of the collagen gel system described herein reveals interesting results regarding the secondary structure of the gel. Circular dichroism of the starting collagen powder revealed a mixture of α-helical structure and random coil, as expected. The α-helical structure is verified by the presence of a positive peak occurring at 220 nm, while the random coil structure is evidenced by a large negative peak occurring at 197 nm. A complete loss of all α-helical structure occurs when the collagen powder is added to the PBS/ethanol solution, as shown in FIG. 11 (collagen solution) by the absence of a positive peak at 220 nm. The collagen solution closely resembles a gelatin gel, which is known to only display a random coil secondary structure. After a complete loss of secondary structure in solution, approximately 60 percent of the α-helical structure is recovered in the collagen gel state. These results were confirmed by FTIR analysis (FIG. 12) which shows a reduction in the amide II peak in the collagen solution compared to the collagen powder and then an increase in the collagen gel compared to the collagen solution.

The methods described herein can create highly concentrated solutions of collagen in water. The solubility of semed S collagen powder in PBS 20×/ethanol is approximately 340 mg/mL. The results described herein shown it is possible to obtain a final collagen concentration of 1.67 g/mL within the collagen gel. The ability to obtain a high concentration of collagen in solution, coupled with the ability to reform α-helical structures within the gel is supportive of fibrillogenesis, or the formation of collagen fibrils.

Other uses for the collagen structures described herein include forming coatings on substrates. For example, medical devices intended for implantation in a human body can be coated with the collagen films or layers using solvent casting, dip coating, spray coating, and the like. Such coating can increase the biocompatibility of any device.

It will be understood that many examples herein are based on the use of collagen; however, other proteins, such as gelatin for example, can be used to achieve the bioadhesives and gels described herein.

The foregoing description of examples has been presented for purposes of illustration and description. It is not intended to be exhaustive or limiting to the forms described. Numerous modifications are possible in light of the above teachings. Some of those modifications have been discussed, and others will be understood by those skilled in the art. The examples were chosen and described in order to best illustrate principles of various examples as are suited to particular uses contemplated. The scope is, of course, not limited to the examples set forth herein, but can be employed in any number of applications and equivalent devices by those of ordinary skill in the art.

What is claimed is:

1. A method for forming a protein structure from a benign solvent comprising:
forming a benign solvent from water, alcohol, and salt;
dissolving a protein in the benign solvent to form a protein solution;
adding alcohol to the protein solution; and
precipitate a protein structure from the protein solution.

2. The method of claim 1, wherein the ratio of alcohol and salt in the benign solvent is about one-to-one by volume.

3. The method of claim 1, wherein the protein is a collagen.

4. The method of claim 3, wherein the collagen is a semed S bovine dermis-derived collagen powder comprising about 95 percent type I collagen and about 5 percent type III collagen.

5. The method of claim 3, wherein the collagen is dissolved in the benign solvent at about 17.5 percent by weight.

6. The method of 1, wherein the alcohol is added to the protein solution by titrating a two-to-one ratio by volume of alcohol to benign solvent.

7. The method of claim 1, wherein the protein structure is a gel.

8. The method of claim 7, further comprising the steps of:
place the gel in water;
stir to form a solution of the gel and water; and
lyophilize the gel and water mixture to form a foam.

9. The method of claim 7, further comprising the steps of:
place the gel in water;
stir to form a solution of the gel and water; and
pour the solution onto a substrate to form a film.

10. The method of claim 9, further comprising the step of washing the film.
